(19) United States
(12) Patent Application Publication
(10) Pub. No.: US 2016/0374785 A1
(43) Pub. Date: Dec. 29, 2016

(54) Method and Apparatus for Facial Visualization and Dental or Operational Planning

(71) Applicants: Uree Goldiner, Tel Aviv, OT (IL); Boris Fridzon, Petah Tikva, OT (IL)

(72) Inventor: Boris Fridzon, Petah Tikva (IL)

(21) Appl. No.: 14/902,560

(22) PCT Filed: Jul. 5, 2014
(86) PCT No.: PCT/IL14/50606

§ 371 Date: Jan. 1, 2016

(30) Foreign Application Priority Data
Jul. 7, 2013 (IL) 227360

(51) Int. Cl. A61C 13/00; G06F 19/00; A61B 90/00

(52) U.S. Cl. CPC A61C 13/0004; A61B 90/37; G06F 19/321; G06F 19/3437; A61B 2034/107

(57) ABSTRACT

A method and apparatus for planning medical treatment, the method comprising: displaying a first image of a patient or a body part, wherein the body part is to be designed or operated on, the first image having first imaging characteristics; displaying a second image of the patient or the body part, the second image having imaging characteristics different from the first imaging characteristics; displaying an image of a model of the body part; receiving a user indication associated with adapting the model for the patient; and outputting a model adapted for the patient, the adapted model based on the model and on the user indication.

### TECHNICAL FIELD

The present disclosure relates to dentistry or facial surgery in general, and to a method and system for visualizing teeth or other facial parts and planning operations or dental treatment, in particular.

### BACKGROUND

A dentist, technician or another oral professional assigned to fix existing teeth of a patient or to design and create one or more false teeth, dental crowns or dental implants faces a challenge.

In order to achieve satisfactory restoration of the patient's mouth, the professional has to make aesthetic and functional diagnosis and planning, which is a complex task. The professional has to take into account a multiplicity of factors, such as but not limited to clinical parameters such as the existing teeth and their size, shape, location and health, teeth to be uprooted, false teeth or dental crowns to be created, the gums structure, size and health, the face including the mouth structure and the smile contour but also other features such as nose and eyes dimensions and distances, temporal and financial considerations, the patient's preferences, or the like. The professional has to consider the face features as they may appear from different angles, hidden areas, feasibility of manufacturing various teeth sets, or the like.

The treatment, including the planning phase is often interdisciplinary and may require the involvement of professionals from multiple disciplines, such as a dentist, a root canal specialist, a technician, a lab worker, assistants or others, which adds extra complexity to the process. In some cases a plastic surgeon may also be involved. Some or all these professional, and optionally the patient as well may have to communicate and provide their input in order to achieve satisfactory results.

### BRIEF SUMMARY

One exemplary embodiment of the disclosed subject matter is a computer-implemented method performed by a computerized device, comprising: displaying a first image of a patient or a body part, wherein the body part is to be designed or operated on, the first image having first imaging characteristics; displaying a second image of the patient or the body part, the second image having imaging characteristics different from the first imaging characteristics; displaying an image of a model of the body part; receiving one or more user indications associated with adapting the model for the patient; and outputting a model adapted for the patient, the adapted model based on the model and on the user indications. Within the method, the body part is optionally one or more teeth. Within the method, one or more may have to be replaced or one or more false teeth may be required to replace the one or more teeth, or a dental implant or a dental crown may be required for the one or more teeth. Within the method, the first image is optionally a frontal image of the patient with the patient's mouth at least partially open and the second image is optionally an image showing the patient's mouth and taken with the patient's mouth opened with an oral retractor. The method may further comprise determining a curve based on the user indications. Within the method the curve is optionally shown on an area of the model that is symmetric to an area on which the user indication is marked. Within the method, the user indications may comprise one or more items selected from the group consisting of: a distance, an angle, and a point. The method may further comprise receiving indications related to other body parts of the patient. The method may further comprise sharing the adapted model with a second user. Within the method the model may ne output to a machine for creating an object in accordance with the model.

Another exemplary embodiment of the disclosed subject matter is an apparatus having a processing unit and a storage device, the apparatus comprising: a user interface adapted for: displaying a first image of a patient or a body part, the first image having first imaging characteristics; displaying a second image of the patient or body part, the second image having imaging characteristics different from the first imaging characteristics; displaying an image of a model of a body part to be designed or operated on; receiving one or more user indications associated with adapting the model for the patient; and a communication component for outputting an adapted model for the patient, the adapted model based on the model and on the user indications. Within the apparatus, the body part is optionally one or more teeth. Within the apparatus, the one or more teeth may be replaced, or it may be required to create one or more false teeth for the one or more teeth, or a dental implant or a dental crown may be required for the one or more teeth. Within the apparatus, the first image is optionally a frontal image of the patient with the patient's mouth at least partially open, and the second image is optionally an image taken with the patient's mouth opened with an oral retractor. The apparatus may further comprise a curve determination component for determining a curve based on the user indications. Within the apparatus, the user interface is optionally adapted to display the curve on a symmetric area of the model that is symmetric to an area on which the user indication is marked. Within the apparatus, the user indications optionally comprise one or more items selected from the group consisting of: a distance, an angle, and a point.

Yet another exemplary embodiment of the disclosed subject matter is a computer program product comprising: a non-transitory computer readable medium; a first computer instruction for displaying a first image of a patient or a body part, wherein the body part is to be designed or operated on, the first image having first imaging characteristics; a second computer instruction for displaying a second image of the patient or the body part, the second image having imaging characteristics different from the first imaging characteristics; a third computer instruction for displaying an image of a model of the body part; a fourth computer instruction for receiving at least one user indication associated with adapting the model for the patient; and a fifth computer instruction for outputting a model adapted for the patient, the adapted model based on the model and on the at least one user indication, wherein said first, second, third, fourth and fifth computer instructions are stored on said non-transitory computer readable medium.

### BRIEF DESCRIPTION OF THE SEVERAL VIEWS OF THE DRAWINGS

The present disclosed subject matter will be understood and appreciated more fully from the following detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure. In the drawings:

FIG. 1 is an illustrative image of an interface showing a patient's face with a grid comprising a symmetry line, in accordance with some exemplary embodiments of the disclosed subject matter;

FIG. 2 shows a part of the image of FIG. 1, with a teeth model shown thereon, in accordance with some exemplary embodiments of the disclosed subject matter;

FIG. 3 shows the image of FIG. 2, with some control points marked by a professional and a tooth outline, in accordance with some exemplary embodiments of the disclosed subject matter;

FIG. 4 shows the image of FIG. 2, with a superimposed under-lips image of the teeth, in accordance with some exemplary embodiments of the disclosed subject matter;

FIG. 5 shows the image of FIG. 4 with some taken measurements, in accordance with some exemplary embodiments of the disclosed subject matter;

FIG. 6 shows a part of the image of FIG. 1, with a smile area indicated, in accordance with some exemplary embodiments of the disclosed subject matter;

FIG. 7 shows the image of FIG. 1, with some additional facial measurements, in accordance with some exemplary embodiments of the disclosed subject matter;

FIG. 8 is a flowchart showing steps in a method for designing body parts or operations thereon, in accordance with some exemplary embodiments of the disclosed subject matter; and

FIG. 9 is a block diagram showing the components of an apparatus for designing body parts or operations thereon, in accordance with some exemplary embodiments of the disclosed subject matter.

### DETAILED DESCRIPTION

The subject matter disclosed below refers to flowcharts and block diagrams describing methods, apparatus, system, or computer program products according to embodiments. It will be appreciated that parts of the block and flowcharts, and combinations of parts, can be implemented by computer program instructions. The computer program instructions may be provided to one or more processors of any computer such as a general purpose computer, special purpose computer, a processor, or other programmable data processing apparatus to produce a machine, such that the instructions, which execute via the processor of the computer or other programmable data processing apparatus, create means for implementing the functions or steps specified in the flowcharts or one or more blocks of the block diagrams.

These computer program instructions may be stored in a non-transient computer-readable medium that can direct a computer or other programmable data processing apparatus to function in a particular manner, such that the instructions stored in the non-transient computer-readable medium produce an article of manufacture including instruction means which implement the functions or acts or blocks specified in the flowchart or block diagram.

The computer program instructions may be loaded onto a device, a computer or other programmable data processing apparatus to cause a series of operational steps to be performed on the computer or other programmable apparatus to produce a computer implemented process such that the instructions which execute on the computer or other programmable apparatus provide processes for implementing the functions, acts, or blocks specified in the flowchart or block diagram.

One technical problem dealt with by the disclosed subject matter is the need to design the structure, size or shape of one or more teeth for a patient, whether for purposes of fixing and reshaping existing teeth, for example by polishing or adding parts, or for making false teeth, dental implants or dental crowns.

The teeth should be of adequate size and shape, so as to match the patient's jaw and gum structure, taking into consideration also other teeth whether such exist or need to be designed as well, the patient's face, smile contour and other factors.

The teeth should also be possible to manufacture and implant. For example, some parts thereof may have to comply with limitations such as minimum dimensions required for ensuring stability available, structures, or the like.

Some of the information may be easily available, such as facial images which may be taken by any digital camera. Other information, such as information related to the existing teeth, the gums structure or the like, may be available from other sources, such as an intra-mouth image taken using a macro lens when the patient's mouth is widely open using an oral retractor.

Manually designing and matching all the data, parameters, options and requirements is a labor-intensive job which requires a lot of measurements, size approximations, experience in selecting an adequate basic set of teeth and in adaptation planning, or the like. The manual designing and fitting process is also highly vulnerable to, mistakes which may require one or more amendment rounds, or which may lead to poor fitting of the teeth to the mouth, functional or aesthetical problems, and in general higher cost or higher inconvenience to the patient or the care giver.

Yet another technical problem dealt with by the disclosed subject matter is the need to share information between multiple care givers. The information may include measures at particular areas, angles, locations, or the like. The measurements have to be taken very accurately, as well as expressed clearly to avoid misunderstandings.

Yet another technical problem dealt with by the disclosed subject matter is the need to assess or predict what the designed teeth would look like once transplanted in the patient's mouth, or after a while. When a tooth is designed to fit into its intended location, it should also be compatible with the other teeth in size, color, shape, angle or other parameters. Even further, a dentist may be fond of the result while the patient may be unhappy for some reason such as a tooth size, tooth color or inconvenience.

One technical solution relates to a method and apparatus for designing teeth for a patient. In some exemplary embodiments, the method comprises presenting to a user, such as a dentist, a technician, an aesthetician or any other professional or even the patient, an image that shows the relevant body part such as the mouth, preferably with its surrounding. For example, a picture of the patient's face may be displayed. If the goal is to design teeth, the picture may be taken when the patient's mouth is open, for example when the patient is smiling. The image may be rotated so as to straighten it around a vertical axis, and one or more lines may be defined on the image, such as a symmetry line, and one or more horizontal or vertical lines which may generate a grid.

The professional may then select an image or model of the relevant part, for example a teeth set. The teeth set may be selected from a collection of teeth sets. The professional may select the set that seems to provide the best match to the teeth of the patient. The professional may select which parts the set will include. For example, when designing one tooth, the professional may select a set comprising all teeth, and leave out all teeth but five which include the tooth to be designed and two teeth on either side.

The model may be resized as required and placed in the appropriate location on the image, for example the face image. The model may be a wire-frame model with transparent or semi-transparent such that the patient's features such as the teeth may be seen under the model. Alternatively, the model may be a three dimensional model in which the projection is displayed as a collection of one or more contours.

The professional may change the outline of each tooth as displayed on the image, to match it as required, for example by moving, adding or deleting control points. In order to keep smooth lines, the tooth outline may be automatically manipulated to represent a Bezier curve, or it may be manually manipulated by the professional. In some embodiments, when designing a tooth, the symmetrical tooth may be automatically manipulated to be symmetric to the designed tooth. However such symmetry may be avoided if required.

Designing one or more teeth may include changing their size, location along the gum, vertical location, or rotation angle. Designing many also include changing the tooth's shape in any required manner.

It will be understood that changing control points or otherwise manipulating the teeth is a design phase which relates to image manipulations, and does not imply changes to actual teeth, unless further actions are taken as described below.

In some embodiments, a different image may be placed on the first image which may provide different information on the same body part. For example, an image of the teeth may be taken using a close-up of the mouth with a macro lens, wherein the patient's mouth may be wide open using an oral retractor, such that the teeth are better exposed, and under-lip view is enabled.

Such image may also be resized and placed in the relevant location over the first image. In the example of teeth designing, the mouth image may be placed over the facial image such that a detailed view of the teeth under the lips is shown in the overall context, e.g., as part of the full face.

The professional may take different measures of the face and/or teeth, gums or the like as part of designing the body part such as the teeth. The professional may also take measures of designed teeth, for example height at a particular area, width, angle, or the like.

In some embodiments, the professional may mark certain areas on the first or the second image, for example the contour of the patient's smile, in order to improve the design, for example make sure a desired part of the teeth is seen when the person is smiling, but the gums are generally covered. In some embodiments, areas such as the smile contour may be determined automatically, for example using edge detection techniques for the facial image.

When the professional has completed the design, or even before that, the professional may store the design, for example the control points, the Bezier curve, the outline of the teeth or the image of the designed teeth, with or without any of the displayed images. The professional may also take measures of the designed areas such as the designed teeth, and may store them together with the designed teeth.

It will be appreciated that although the examples above relate to teeth, the disclosure is not limited to teeth and may be used for designing other parts such as the nose or eyes area before a surgery. The system and method may be used for planning aesthetic as well as functional operations.

One technical effect of the disclosed subject matter relates to providing an easy, intuitive and efficient apparatus for designing operations such as aesthetical surgeries, or designing parts such as teeth. The method and apparatus provide for identifying, measuring or evaluating the clinical patient parameters, such that the design may take thee parameters into account.

The method and apparatus provide for combining data from multiple sources such as different types of images, and superimposing them to achieve better results. For example, making sure that the designed teeth are compatible with the gums size and structure, as seen in the intra-mouth image, while being compatible with the face as a whole, the smile, or other factors.

The system and method provide for selecting from multiple predetermined sets of teeth or other parts, to be used as a basis for design.

Thus, the disclosed method and apparatus provide for diagnosing conditions, identifying risks and predicting the treatment outcomes in an efficient and effective manner.

Another technical effect of the disclosed subject matter relates to communicating and sharing the design among multiple professionals. For example, after a dentist designed one or more teeth, a root canal specialist or a technician may review or enhance the design. Even further, a technician or a laboratory may receive the design for molding or otherwise creating one or more false teeth or dental crowns as designed.

Yet another technical effect of the disclosed subject matter relates to sharing the design with the patient, by showing the patient what the body part is expected to look like when transplanted or operated. In some embodiments, the expected look is the final result that will be assumed after the initial swelling or other side effects disappear, and the tooth assumes its final position.

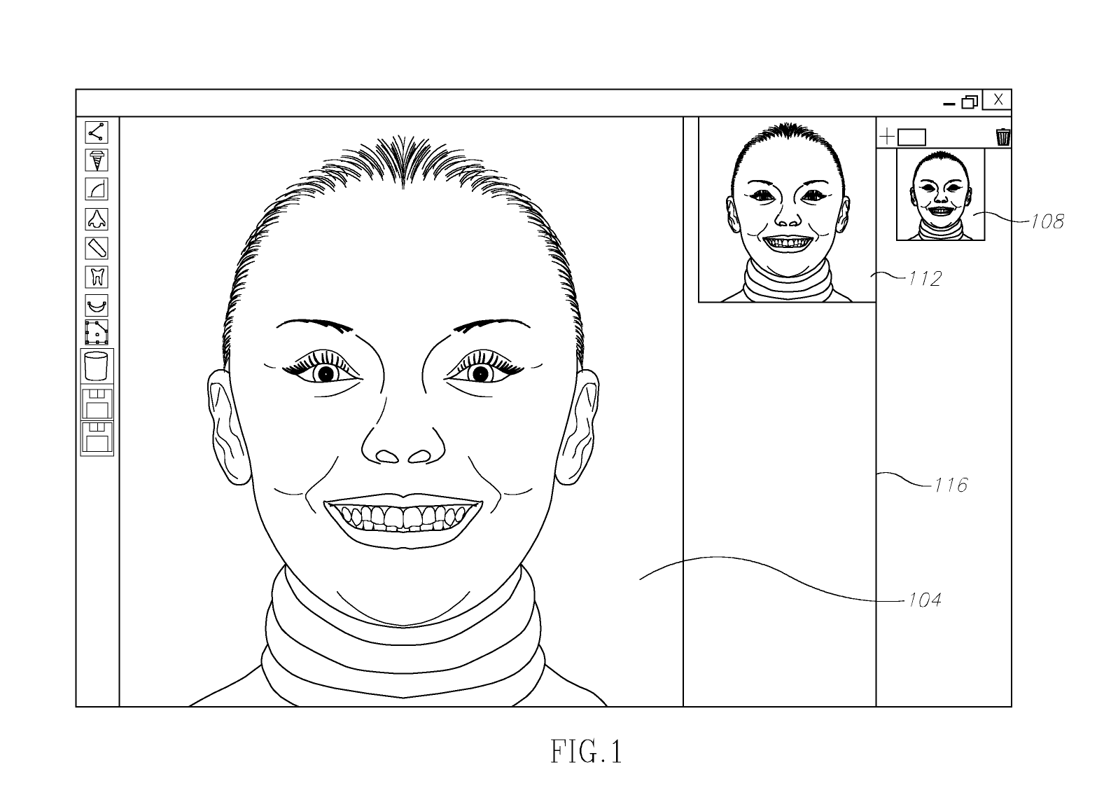

Referring now to FIGS. 1-7, showing a user interface of an apparatus for designing teeth for a patient. A user of the apparatus may be a professional such as a dentist, a teeth technician, or the like.

Referring now to FIG. 1, showing a main image area 104 displaying a frontal image of a patient. The patient is smiling so that her teeth are visible. However, it will be appreciated that the way the patient's picture is taken depends on the purpose: for teeth designing it may be preferable that the patient's mouth is open, while in nose surgery planning this may not be required.

Area 108 on the right hand side of the user interface shows smaller images from which the user can choose which image to show in area 104.

Area 112 shows a small version of the image displayed in area 104. Area 112 always shows the full image, on which the zoomed-in part as shown in area 104 is marked, to provide the user with orientation of which part of the full image is currently displayed in area 104.

The image in area 014 has thereon a symmetry line 116 and additional lines constituting a grid.

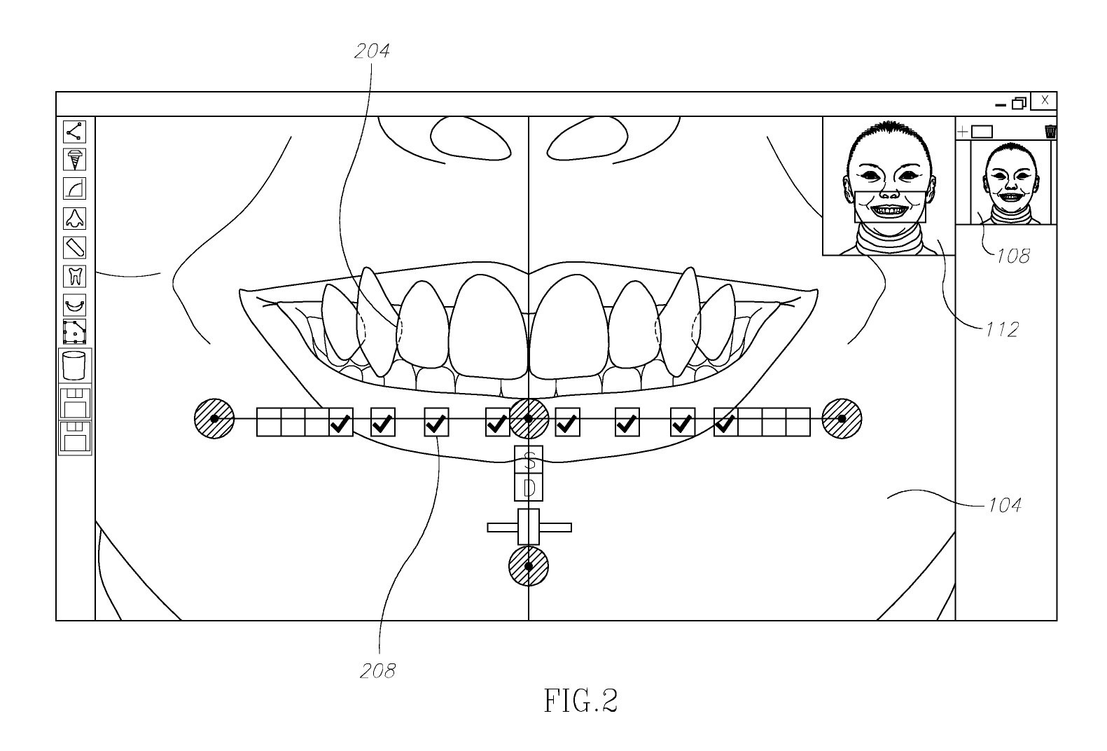

Referring now to FIG. 2, showing area 104 with a zoom-in on the mouth of the patient shown in area 104 of FIG. 1. The shown part is indicated by a rectangle drawn around the mouth area on the image shown in area 112.

Also shown in area 104 is an image 204 of a teeth model, which may have been selected by the user from a multiplicity of models. Image 204 may have been moved, rotated, or resized to fit the size and location of the patient's teeth as displayed. The model may be displayed in a semi-transparent manner so that the patient's teeth are also visible, such that the model may be adapted for the specific patient.

Also shown is a legend 208 from which the user may select which teeth should be part of the model. For example, if a person has a certain tooth and it is not planned to reconstruct that tooth, then the tooth may be unchecked and not be part of the model. The user may also select or change additional parameters.

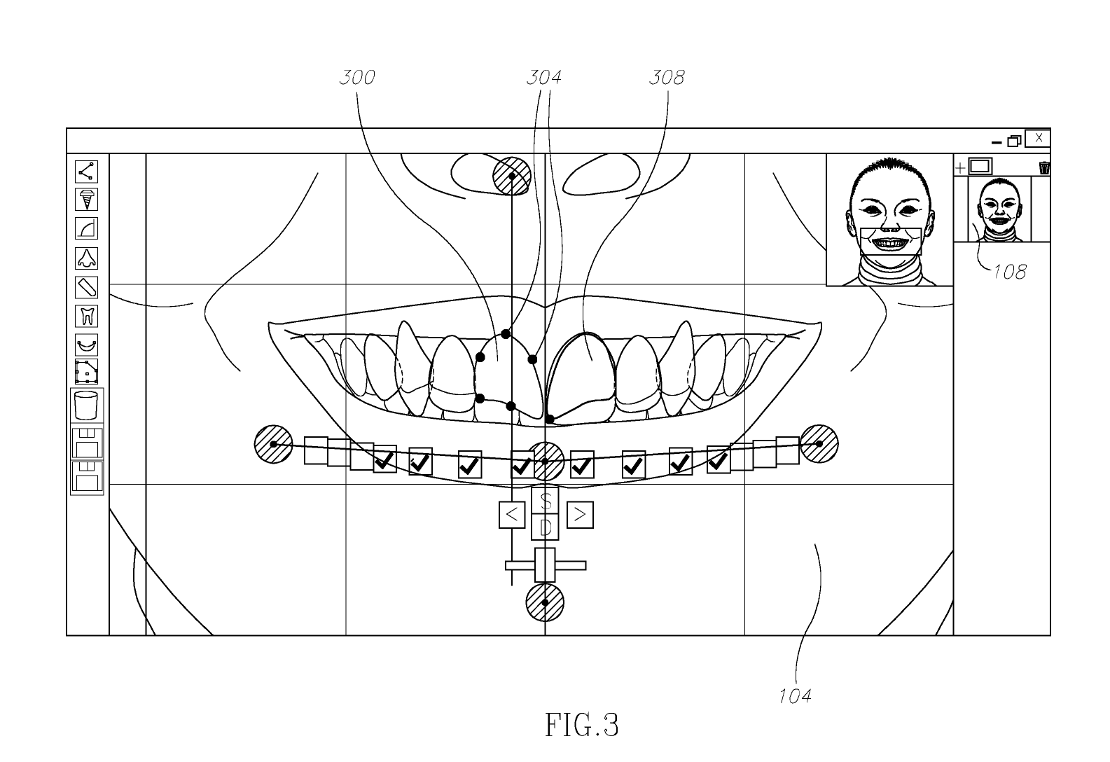

Referring now to FIG. 3, showing markers 304 as indicated by the user on tooth 300, in order to design its boundary. The boundary may be designed according to the existing tooth as seen through the model image, to fill missing parts of the existing tooth, to create a brand new false tooth or dental crown in accordance with an existing symmetrical tooth, or the like. Symmetrical tooth 308 may be marked with a curved contour 312, determined according to points 304 indicated on the symmetrical tooth. The contour may be determined as a Bezier curve or any other preferably smooth curve connecting control points 304 or the symmetrical points.

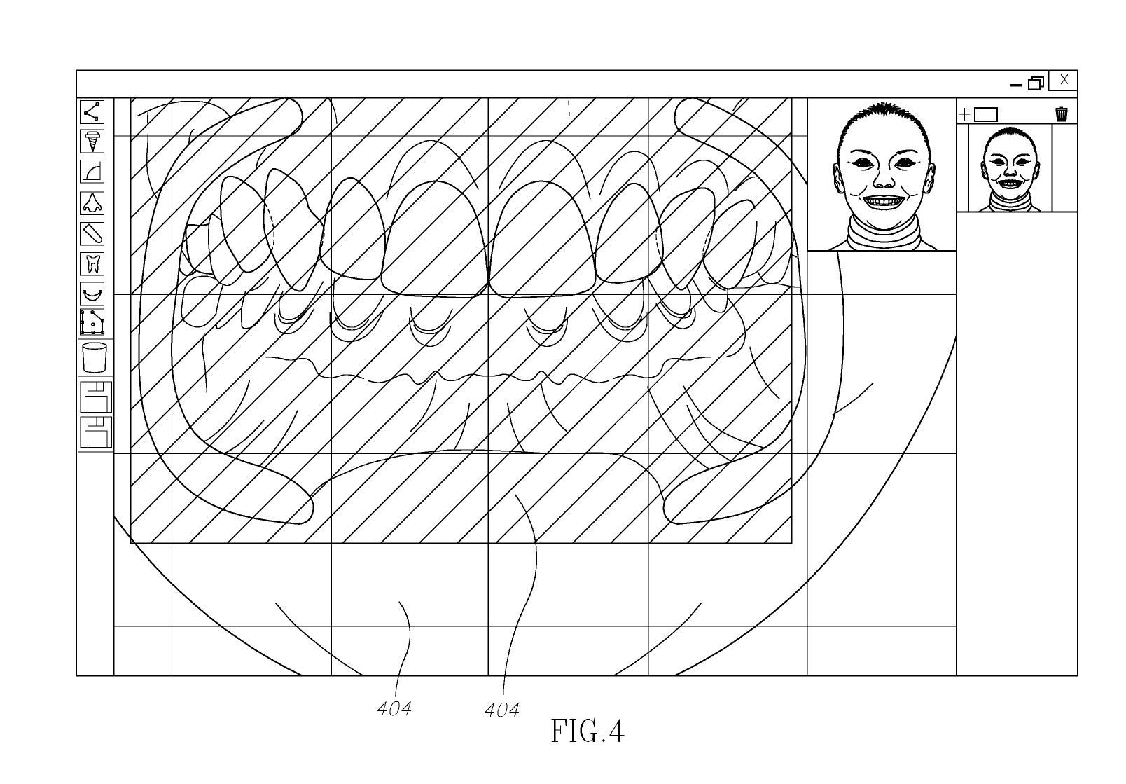

Referring now to FIG. 4, showing the mouth area of the patient shown in area 104 of FIG. 1, with an additional image 400 which may have been taken by a macro lens when the patient's mouth is opened using an oral retractor. Image 400 shows also the teeth parts behind the lips, and thus provides a fuller image of the teeth. Image 400 may be adjusted in size and location to the patient's image and superimposed thereon. Image 400 may hide the relevant area of the patient's image, or may be transparent to some degree. Model 300 may still be shown to facilitate designing the teeth in view of image 400.

It will be appreciated that image 400 may be laid over only part of the patient's image, such that other parts for example chin 404 or other areas are visible (although these parts, for example the patient's eyes, may be out of the field of view is changed), in order to provide an overall view of the teeth within the context of the face.

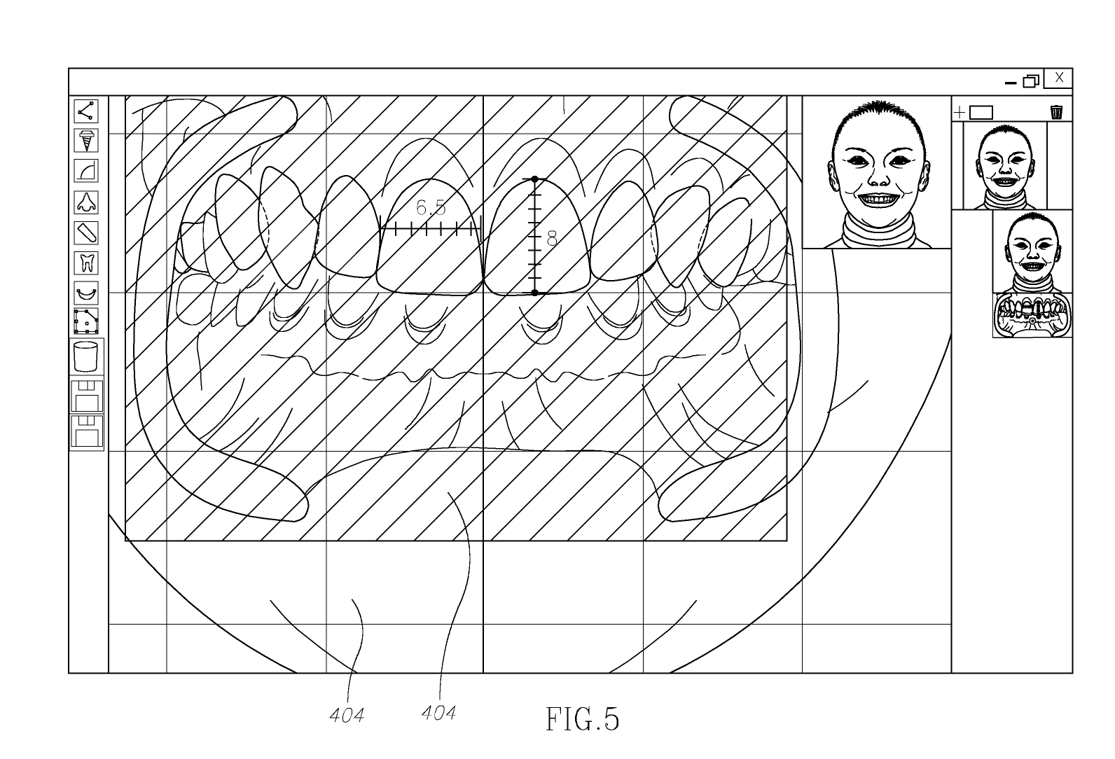

Referring now to FIG. 5, showing image 400 together with the teeth model, and with some indications thereon, such as the measurements showing required dimensions of the designed tooth.

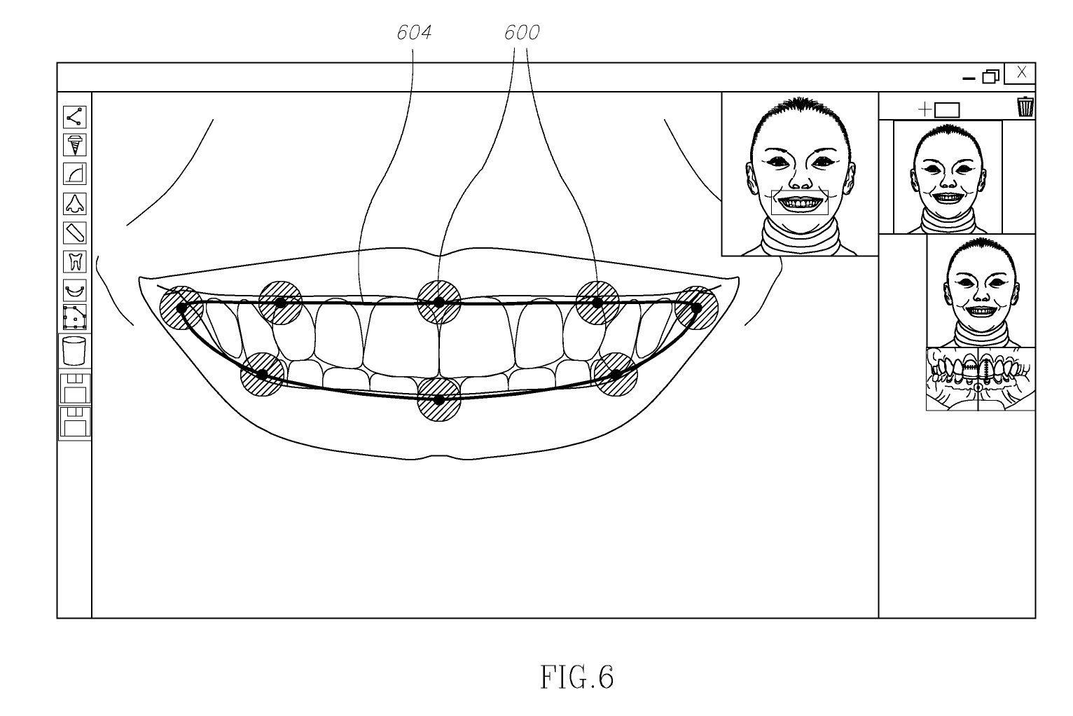

Referring now to FIG. 6, showing a part of image area 104 with control points 600 and curve 604, which indicates the smile area of the patient. Marking the smile area enables to design the teeth so that they are aesthetically compliant with the smiles, for example a portion of the teeth is visible when the person smiles but the gums are hidden. It will be appreciated that control points 600 and curve 604 may mark other parts or effects in a patient's face or body, and not necessarily the smile area.

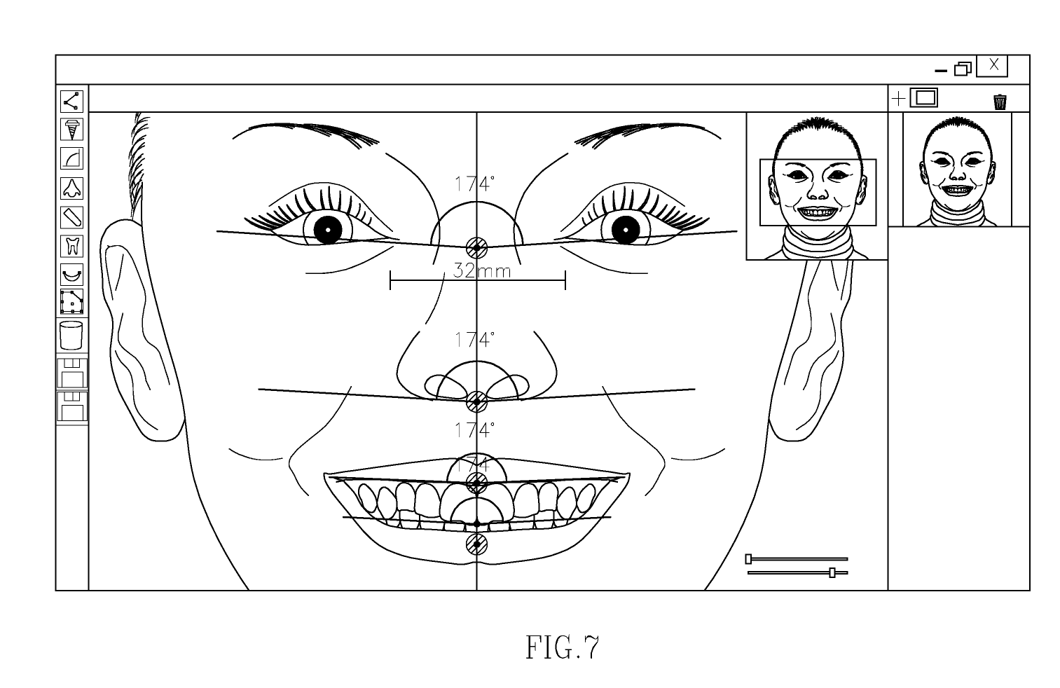

Referring now to FIG. 7, showing a significant part of the patient's image, with different measurements, such as different distances, angles, or the like. Determining the measurements may provide for better adjustment of teeth parameters, such as teeth angles, sizes, or the like.

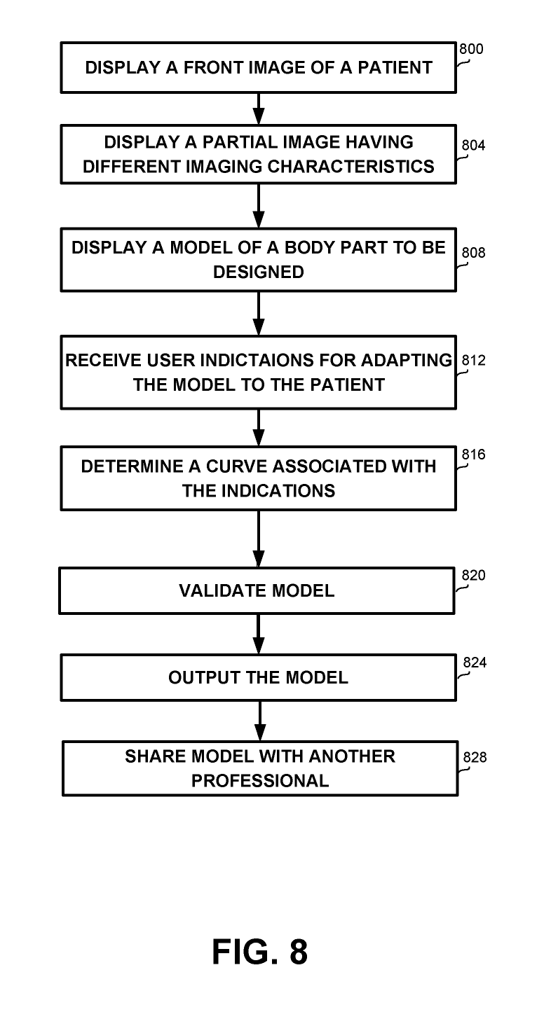

Referring now to FIG. 8, showing a flowchart of steps in a method for designing a body part such as teeth or an operation on a body part.

On step 800 an image of the body part of the patient may be displayed on a display device. The image may show the patient in full, or a part thereof such as a facial image. In some embodiments, such as dental applications, the first image may be an image of the patient's face, with the patient smiling so that the patient's teeth are visible.

On step 804, a second image of the patient may be displayed, wherein the second image may have different characteristic, such as being partial to the first image, being taken with different equipment such as a different lens, being taken with special handling of the body part. For example in the dental application, the second image may be taken by a macro lens with the patient's mouth opened with an oral retractor. The second image may be displayed on top of the first image, after having been resized, rotated, moved or otherwise adapted. The top image may be displayed with a degree of transparency so that the bottom image is also visible.

On step 808, an image of a model of the body part to be designed or to be operated on may be displayed. The model may be selected from multiple models according to a user's choice, and may be replaced if the user feels it is suboptimal. The model image may include feature indications and outlines, such as control points, teeth outlines, or other indicators. The model may be configured, for example indicated to comprise only some teeth, in accordance with the design task.

The model image may be displayed such that only the contours are visible, while internal areas are transparent, such that the body part can be best adapted in accordance with the first and the second images which may be visible in addition to the model image.

The model or any part thereof may be moved, rotated, resized or otherwise manipulated to fit the first or the second images as displayed on the display device.

It will be appreciated that the first image, the second image and the model image may be displayed in any desired temporal order (e.g. any image or model may be displayed prior to or after another one), and in any vertical order (e.g., any image or model may be displayed above or below any other one such that it may fully or partially hide or expose it).

On step 812, user indications related to the model may be received, such as size measurements, control point marking, or other indications. The user may also mark which parts of the model are required, for example selecting only some teeth from a model comprising a full set of teeth.

On step 816, additional manipulations may be performed, such as determining a curve based on points marked on the model image. Manipulations may also include manipulations to other teeth in the model. For example, in response to the user marking control points on one tooth, adding a curve line connecting corresponding control points on a symmetrical tooth.

On optional step 820 the model may be validated. Such validation may include professional validation, for example adherence with medical standards and principles such as tooth structure and strength, administrative principles, such as verifying that the patient approved the output before manufacturing, or the like. Additional validation may relate to color selection, for example verifying that color match between a new tooth and existing ones was determined according to at least a predetermined number of images taken under different lightning conditions.

On optional step 824 the model may be output, for example to a file, a printer, a plotter, another display, a manufacturing device, or the like. In some embodiments, the model may be output to a laboratory or a machine for molding or otherwise generating objects to be used as one or more false teeth or dental crowns in accordance with the model.

On optional step 828 the model may be shared with another professional or institute such as a technician, a laboratory or another expert. The model may be shared via a file sharing system, e-mail, a social network or any other platform and communication channel.

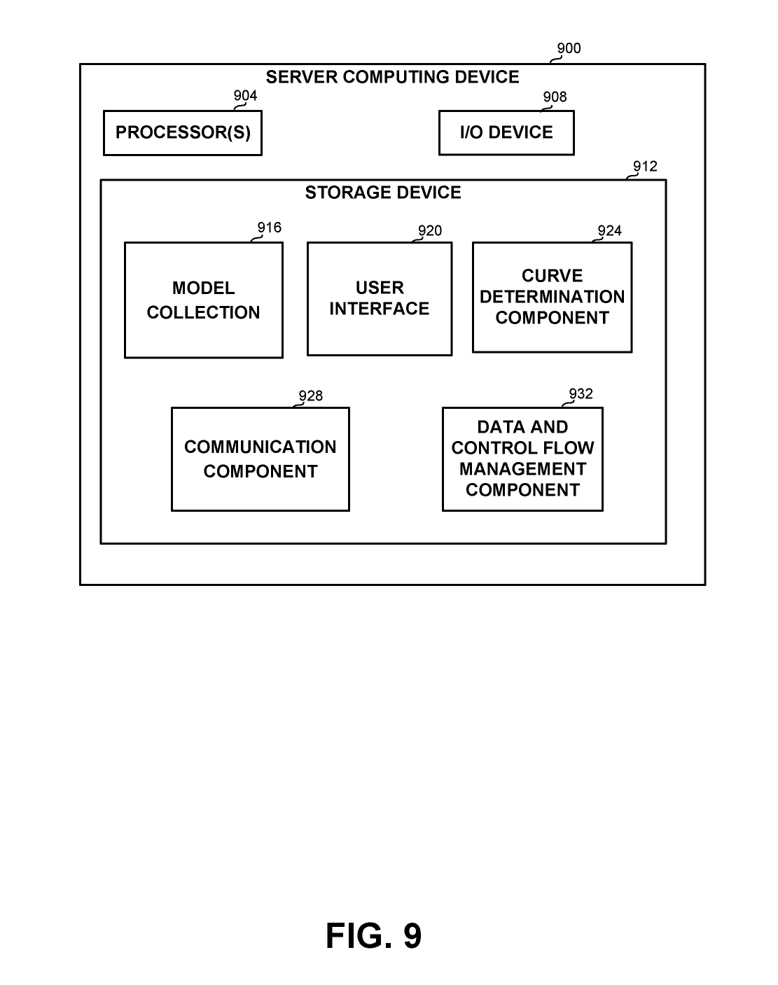

Referring now to FIG. 9, showing a block diagram of an apparatus for designing a body part such as teeth or an operation on a body part.

The apparatus may comprise a computing platform 900. Computing platform 900 may comprise a processor 904. Processor 904 may be a Central Processing Unit (CPU), a microprocessor, an electronic circuit, an Integrated Circuit (IC) or the like. Alternatively, computing platform 900 can be implemented as firmware writen for or ported to a specific processor such as digital signal processor (DSP) or microcontrollers, or can be implemented as hardware or configurable hardware such as field programmable gate array (FPGA) or application specific integrated circuit (ASIC). Processor 904 may be utilized to perform computations required by computing platform 900 or any of it subcomponents.

In some embodiments, computing platform 900 may comprise an input-output (I/O) device 908 such as a terminal, a display, a keyboard, a camera, a sensor, a touch screen, an input device or the like, used to interact with the system, to invoke the system and to receive results.

In some exemplary embodiments, comptuing platform 900 may comprise one or more storage devices such as storage device 912. Storage device 912 may be persistent or volatile. For example, storage device 912 can be a Flash disk, a Random Access Memory (RAM), a memory chip, an optical storage device such as a CD, a DVD, or a laser disk; a magnetic storage device such as a tape, a hard disk, storage area network (SAN), a network attached storage (NAS), or others; a semiconductor storage device such as Flash device, memory stick, or the like. In some exemplary embodiments, storage device 912 may retain program code operative to cause processor 904 to perform acts associated with the development platform.

Storage device 912 may comprise or be in association with one or more file systems, responsible for maintaining the files associated with the platform, including creating, saving updating, or the like.

Storage device 912 may comprise a model collection 916, comprising one or more models of body parts such as teeth, which may serve as basis for designing operations or and creating objects to be used as body parts for particular patients.

Storage device 912 may also comprise an application implementing a method as disclosed in association with FIG. 8 above. The application may comprise various components, which may be implemented as one or more sets of interrelated computer instructions, executed for example by processor 904 or by another processor. The components may be arranged as one or more executable files, dynamic libraries, static libraries, methods, functions, services, or the like, programmed in any programming language and under any computing environment.

The components may include user interface 920 which provides a framework for displaying a first image and a second image wherein the first image and the second image have different imaging characteristics. User interface 920 may also provide the user with tools for resizing, moving or rotating one or more of the images so that they match; for selecting a model, enabling the user to move, resize or rotate an image of the model, and to manipulate the model for example by marking points; for receiving selections and indications from the user, and additional actions.

Storage device 912 may also comprise a curve determination component 924, for determining a curve such as a Bezier curve based upon control points indicated by a user, upon control points symmetrical to points indicated by the user, or the like. The curve may serve as an outline for a body part such as a tooth.

Storage device 912 may further comprise a communication component 928 for outputting or sharing the model, optionally with changes introduced by a user. The model may be output to a file, a machine, or shared with another user such as the patient, or another professional. Sharing may be done using a file system, a sharing application, a social network, a distributed application or the like. In some embodiments, the application may also provide for collaboration in which multiple users can collaborate by working on the same model simultaneously. In such embodiments, the application may be equipped with a chat tool for exchanging ideas, opinions and explanations.

Storage device 912 may comprise data and control flow management component 932 for managing the flow of information and control between other components, for example retrieving models, storing images, controlling the image display, generating curves, sharing output, or the like.

The disclosed method and apparatus provide for efficient and effective design of body parts or operations thereon. By superimposing images showing different aspects and angles of the user's body or parts thereof, a better match can be achieved. The process is made more efficient by using readymade models from which a user can select a suitable model, such that as little as possible changes are required.

The method and apparatus may provide for designing teeth or for fixing existing teeth for example by polishing or adding parts.

It will be appreciated that the disclosed method and apparatus may be combined with additional sources, such as X-ray images, panoramic images, or the like. Such sources may be used in conjunction with other sources, for example by showing superimposed sources or elements therefrom.

The method and apparatus may also provide for planning other operations, such as nose operations, in which different images, such as a frontal image and an internal image taken using electro optical fibers may be combined, and may be further combined with existing models.

The flowchart and block diagrams in the figures illustrate the architecture, functionality, and operation of possible implementations of systems, methods and computer program products according to various embodiments of the present disclosure. In this regard, each block in the flowchart and some of the blocks in the block diagrams may represent a module, segment, or portion of program code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As will be appreciated by one skilled in the art, the disclosed subject matter may be embodied as a system, method or computer program product. Accordingly, the disclosed subject matter may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product embodied in any tangible medium of expression having computer-usable program code embodied in the medium.

Any combination of one or more computer usable or computer readable medium(s) may be utilized. The computer-usable or computer-readable medium may be, for example but not limited to, any non-transitory computer-readable medium, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CDROM), an optical storage device, a transmission media such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to wireless, wireline, optical fiber cable, RF, and the like.

Computer program code for carrying out operations of the present disclosure may be written in any combination of one or more programming. languages, including an object oriented programming language such as Java, Smalltalk, C++ or the like, conventional procedural programming languages, such as the “C” programming language or similar programming languages, scripting languages such as Pert, Python, Ruby, or any other programming language. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

What is claimed is:

1. A computer-implemented method performed by a computerized device, comprising:
displaying a first image of a patient or a body part, wherein the body part is to be designed or operated on, the first image having first imaging characteristics;
displaying a second image of the patient or the body part, the second image having imaging characteristics different from the first imaging characteristics;
displaying an image of a model of the body part, wherein the model is selected by a user;
receiving at least one user indication associated with adapting the model for the patient; and
outputting a model adapted for the patient, the adapted model based on the model and on the at least one user indication.

2. The method of claim 1, wherein the body part is at least one tooth.

3. The method of claim 2, wherein the at least one tooth is to be replaced or at least one tooth for which it is required to create a false tooth, a dental implant or a dental crown.

4. The method of claim 2, wherein the first image is a frontal image of the patient with the patient's mouth at least partially open and the second image is an image showing the patient's mouth and taken with the patient's mouth opened with an oral retractor.

5. The method of claim 1, further comprising determining a curve based on the at least one user indication.

6. The method of claim 5, wherein the curve is shown on an area of the model that is symmetric to an area on which the user indication is marked.

7. The method of claim 1, wherein the at least one user indication comprises at least one item selected from the group consisting of: a distance, an angle, and a point.

8. The method of claim 1, further comprising receiving indications related to other body parts of the patient.

9. The method of claim 1, further comprising sharing the adapted model with a second user.

10. The method of claim 1, wherein the model is output to a machine for creating an object in accordance with the model.

11. An apparatus having a processing unit and a storage device, the apparatus comprising:
a user interface adapted for:
displaying a first image of a patient or a body part, the first image having first imaging characteristics;
displaying a second image of the patient or body part, the second image having imaging characteristics different from the first imaging characteristics;
displaying an image of a model of a body part to be designed or operated on, wherein the model is selected by a user;
receiving at least one user indication associated with adapting the model for the patient; and
a communication component for outputting an adapted model for the patient, the adapted model based on the model and on the at least one user indication.

12. The apparatus of claim 11, wherein the body part is at least one tooth.

13. The apparatus of claim 12, wherein the at least one tooth is to be replaced or at least one tooth for which it is required to create a false tooth, a dental implant or a dental crown.

14. The apparatus of claim 12, wherein the first image is a frontal image of the patient with the patient's mouth at least partially open, and the second image is an image taken with the patient's mouth opened with an oral retractor.

15. The apparatus of claim 11, further comprising a curve determination component for determining a curve based on the at least one user indication.

16. The apparatus of claim 15, wherein the user interface is adapted to display the curve on a symmetric area of the model that is symmetric to an area on which the user indication is marked.

17. The apparatus of claim 11, wherein the user indications comprises at least one item selected from the group consisting of: a distance, an angle, and a point.

18. A computer program product comprising:
a non-transitory computer readable medium;
a first computer instruction for displaying a first image of a patient or a body part, wherein the body part is to be designed or operated on, the first image having first imaging characteristics;
a second computer instruction for displaying a second image of the patient or the body part, the second image having imaging characteristics different from the first imaging characteristics;
a third computer instruction for displaying an image of a model of the body part, wherein the model is selected by a user;
a fourth computer instruction for receiving at least one user indication associated with adapting the model for the patient; and
a fifth computer instruction for outputting a model adapted for the patient, the adapted model based on the model and on the at least one user indication,
wherein said first, second, third, fourth and fifth computer instructions are stored on said non-transitory computer readable medium.
